(19) United States
(12) Patent Application Publication
(10) Pub. No.: US 2024/0346818 A1
(43) Pub. Date: Oct. 17, 2024

(54) INSPECTION DEVICE

(71) Applicant: HITACHI, LTD., Tokyo (JP)

(72) Inventors: Takafumi MIWA, Tokyo (JP); Sayaka KURATA, Tokyo (JP); Shinichi MATSUBARA, Tokyo (JP); Yuta IMAI, Tokyo (JP)

(21) Appl. No.: 18/292,978

(22) PCT Filed: Sep. 21, 2022
(86) PCT No.: PCT/JP2022/035155

§ 371 Date: Jan. 29, 2024

(30) Foreign Application Priority Data
Oct. 8, 2021 (JP) 2021-166263

(51) Int. Cl. G06V 10/98; G06T 7/00; G06V 10/25; G06V 10/88

(52) U.S. Cl. CPC G06V 10/993; G06T 7/0014; G06V 10/25; G06V 10/88; G06T 2207/10061

(57) ABSTRACT

The purpose of the present invention is to provide an inspection device that can determine the reliability of an image feature value in a target region of an observation image of a sample. This inspection device extracts a first feature value from a target region including an inspection target in an observation image of a sample, extracts a second feature value from a reference region other than the target region in the observation image, and compares the first feature value and the second feature value, thereby calculating the reliability of the first feature value (see FIG. 1).

### TECHNICAL FIELD

The present disclosure relates to an inspection device that inspects a sample using an observed image of the sample.

### BACKGROUND ART

A method is reported that acquires an image of a sample observed by using a device such as microscope, and that inspects the sample according to the image. Non Patent Literature 1 below describes a method that inspects a number of particles at an inspection position, by comparing an image feature at the inspection position with a feature (number of particles) of the background, within the observed image acquired by electron microscope.

### CITATION LIST

### Non Patent Literature

Non Patent Literature 1: Journal of Pharmaceutical and Biomedical Analysis 196 (2021) 113924

### SUMMARY OF INVENTION

### Technical Problem

When using the inspection method above, if there is no significant difference between the image feature at the inspection position and the image feature at a reference position, it is difficult to precisely identify the number of particles at the inspection position, or the like. Then the inspection accuracy will be decreased.

In order to show that the difference is significant, it may be conceivable to acquire a plurality of observed images, and to compare features at the inspection position and at the reference position, within each of the observed images. However, even if a plurality of observed images is acquired, such observed images are not always all appropriate for comparison. For example, due to preparing process of the sample, a defective image may occur by factors such as impurities mixed into the inspection position or into the reference position. If such defective image is used, the reliability of image feature is decreased, and then no reliable result will be acquired in the inspection using image features. If the number of images is increased, the reliability of inspection result will be improved to some extent. On the other hand, the inspection throughput will be decreased.

This disclosure is made in the light of the technical problem above. The objective of this disclosure is to provide an inspection device that can determine a reliability of an image feature at a target area of an observed image of a sample.

### Solution to Problem

An inspection device according to this disclosure: extracts a first feature from a target area including an inspection target within an observed image of a sample; extracts a second feature from a reference area other than the target area within the observed image; and calculates the reliability of the first feature by comparing the first feature with the second feature.

### Advantageous Effects of Invention

With the inspection device according to this disclosure, it is possible to determine a reliability of an image feature at a target area of an observed image of a sample. Other aspects such as problems, configurations, or advantages of this disclosure will be apparent by referring to the embodiments below.

### BRIEF DESCRIPTION OF DRAWINGS

FIG. 1 is a configuration diagram of an inspection device 1 according to an embodiment 1.

FIG. 2 is a diagram explaining a significant difference in an immunochromatography inspection kit.

FIG. 3 is a side cross-sectional diagram illustrating a configuration example of an image acquiring device 10.

FIG. 4 is a flowchart that explains a conventional procedure for performing inspection using an observed image of immunochromatography inspection kit.

FIG. 5 is a flowchart that explains a behavior of the inspection device 1.

FIG. 6 illustrates a specific example of S503.

FIG. 7 is a schematic diagram that explains a specific example of S502.

FIG. 8 is an example of user interface provided by an input/output device 30.

FIG. 9 is an example of user interface provided by an input/output device 30.

FIG. 10 is an example of data stored in a storage 28.

FIG. 11 is a flowchart that explains a behavior of the inspection device 1 according to an embodiment 2.

FIG. 12 is a diagram illustrating another example of a sample 40 and its image feature.

### DESCRIPTION OF EMBODIMENTS

### Embodiment 1

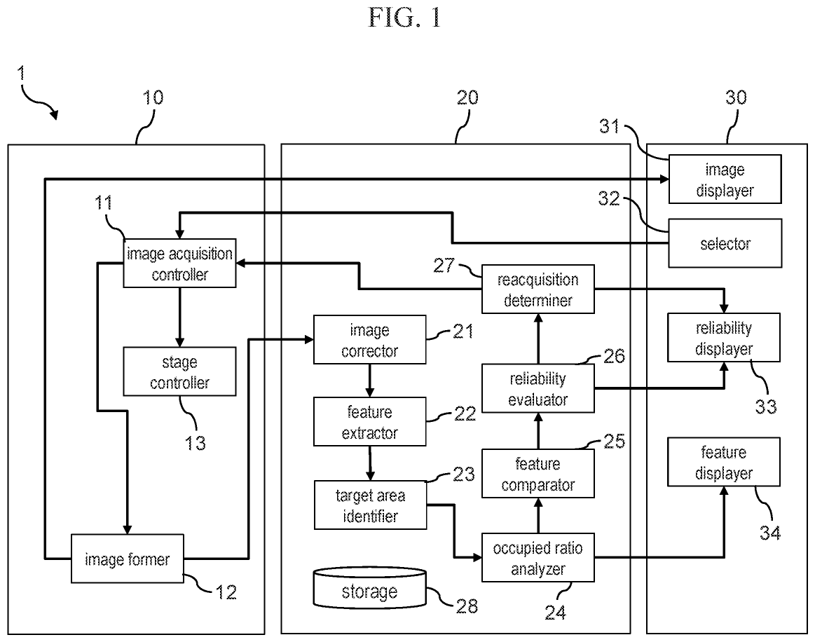

FIG. 1 is a configuration diagram of an inspection device 1 according to an embodiment 1 of this disclosure. The inspection device 1 is a device that inspects a sample using an observed image of the sample. The inspection device 1 includes an image acquiring device 10, an evaluating device 20, and an input/output device 30. The sample is, for example, an immunochromatography inspection kit. An immunochromatography inspection kit is a device that detects antigen or antibody by providing a sample liquid, containing the antigen or the antibody to be inspected, onto a plate formed by such as porous material. Marker particles contained in the sample liquid or in the plate flow on the surface of the plate or flow into the plate along with the sample liquid. Then it is possible to visually detect the antigen or the antibody according to changes in color caused on the marker particles which are captured by capturing antibodies on the plate. The marker particles include various conductive or insulating particles, such as metal particle, latex particle, or silica particle. It is also possible to perform an inspection using image features of the inspection kit, instead of using color changes on the marker particles. For example, the feature at an inspection position and the feature at a reference position other than the inspection position are compared with each other. The reference position is a position where it is assumed that there is no marker particle (or there is sufficiently little marker particle). Using the feature at the reference position as a reference, it is possible to inspect a number of marker particles (i.e. amount of antigen or of antibody) at the inspection position by comparing the reference with the feature at the inspection position. When using the immunochromatography inspection kit, the sample can be a plate that holds a liquid containing marker particles. However, the sample is not limited to it.

The image acquiring device 10 acquires an observed image of the sample. Instead of visually checking the sample, the sample is inspected by analyzing image features of the sample. For example, by evaluating an image feature that correlates with the number of marker particles, it is possible to acquire an inspection result more precise than that of visually checking the marker particles. Specific configurations of the image acquiring device 10 will be described later.

The evaluating device 20 evaluates a reliability of features of the observed image acquired by the image acquiring device 10. The reliability mentioned here is an indicator that indicates whether there is a significant difference between an image feature of a target area where the inspection target (in the case of immunochromatography inspection kit, marker particle) exists and an image feature of a reference area (in the case of immunochromatography inspection kit, an area on the inspection kit where there is no or very little marker particle). An example of the significant difference and of the reliability will be described later.

The evaluating device 20 includes an image corrector 21, a feature extractor 22, a target area identifier 23, an occupied ratio analyzer 24, a feature comparator 25, a reliability evaluator 26, a reacquisition determiner 27, and a storage 28. The storage 28 can be configured by a storage device storing data. Other functional units can be configured by hardware such as circuit device implementing those functions, or can be configured by software implementing those functions executed by processors such as CPU (Central Processing Unit). Behaviors of these functional units will be described later.

The input/output device 30 is a device that displays a processed result by the evaluating device 20. The input/output device 30 is also a device to which a user inputs instructions that are provided to the evaluating device 20 and to the image acquiring device 10. The input/output device 30 includes an image displayer 31, a selector 32, a reliability displayer 33, and a feature displayer 34. Behaviors of these functional units will be described later.

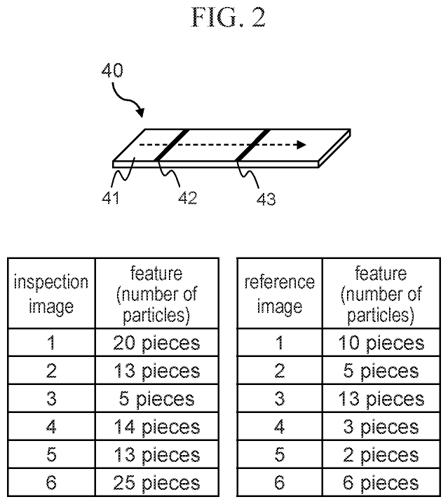

FIG. 2 is a diagram explaining a significant difference in the immunochromatography inspection kit. In the immunochromatography inspection kit (sample) 40, an inspection position 42 (target area) and a reference position 43 (reference area) are configured on a porous plate 41. A liquid containing an inspection target such as antigen or antibody flows in the arrow direction on the plate 41. When the inspection target combines with marker particles such as metal particles, it is possible to inspect the number of inspection targets by counting the marker particles.

In this embodiment, in order to improve the inspection accuracy, the amount of inspection target at the inspection position 42 is determined by comparing an image feature at the inspection position 42 with an image feature at the reference position 43, instead of counting the number of marker particles themselves. It is assumed that there is no or very little inspection target particles at the reference position 43. The image feature is a number of inspection target particles or an amount corresponding to such number. For example, the image feature may be such as an area size or a density of the inspection target particle.

An actual image feature at the reference position 43 is not always 0, and may be varied for each of observed images. Accordingly, there may be no significant difference of image feature between the inspection position 42 and the reference position 43. In the example shown in FIG. 2, the first observed image at the inspection position 42 includes 20 pieces of inspection target particles, and the first observed image at the reference position 43 includes 10 pieces of inspection target particles. The second or later observed images each include inspection target particles as shown in FIG. 2. If these features have no significant differences, when inspecting the observed image at the inspection position 42 by using the observed image at the reference position 43 as a reference, a correct inspection result may not be acquired.

Accordingly, in the embodiment 1, a reliability of image features at the inspection position 42 is determined by evaluating whether there is a significant difference between the image feature at the inspection position 42 and the image feature at the reference position 43.

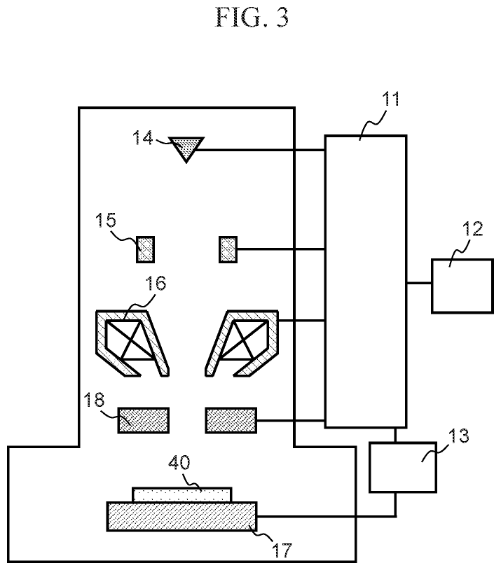

FIG. 3 is a side cross-sectional diagram illustrating a configuration example of the image acquiring device 10. The image acquiring device 10 is a device that acquires an observed image of a sample 40 by irradiating the sample 40 with an electron beam. The image acquiring device 10 is a kind of charged particle beam device. The image acquiring device 10 includes an image acquisition controller 11, an image former 12, a stage controller 13, an electron source 14, a deflector 15, a lens 16, a stage 17, and a detector 18.

The electron source 14 emits an electron beam. The deflector 15 deflects the direction of the electron beam. The lens 16 irradiates the electron beam onto the sample 40. The sample 40 is placed on the stage 17. The detector 18 detects secondary particles generated from the sample 40 by irradiating the electron beam onto the sample 40. The detector 18 outputs a detection signal that represents an intensity of the secondary particle. The image former 12 creates an observed image of the sample 40 using the detection signal. The stage controller 13 controls the stage 17. The image acquisition controller 11 controls overall behaviors of the image acquiring device 10.

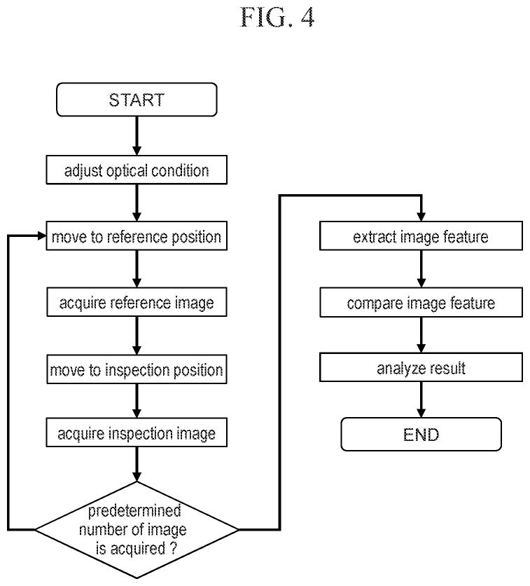

FIG. 4 is a flowchart that explains a conventional procedure for performing inspection using an observed image of immunochromatography inspection kit. In the conventional procedure, observed images are acquired at the reference position 43 and at the inspection position 42 respectively. After acquiring a predetermined number of observed images, the image features at each position are compared with each other, and then a comparison result is analyzed. These procedures are performed manually by a person who operates the device. In this procedure, it is not evaluated whether there is a significant difference between the image feature at the reference position 43 and the image feature at the inspection position 42. Therefore, the inspection result may not be reliable.

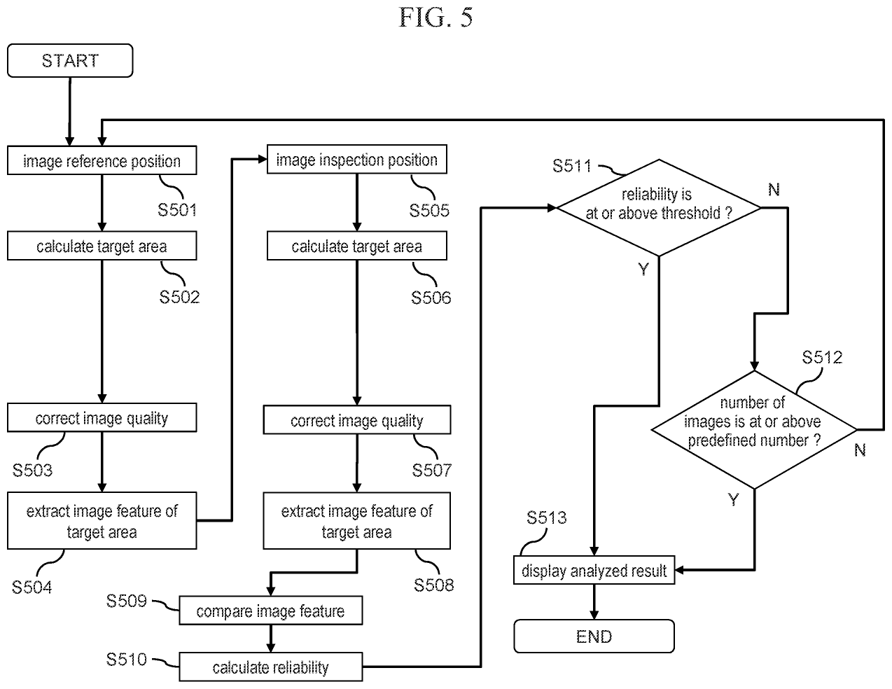

FIG. 5 is a flowchart that explains a behavior of the inspection device 1. This flowchart includes a behavior of the inspection device 1 for determining a reliability of image feature at the inspection position 42. Hereinafter, each step in FIG. 5 will be described.

### (FIG. 5: Step S501)

The stage controller 13 moves the stage 17 to a position where the electron beam is irradiated onto the reference position 43. The image acquisition controller 11 adjusts imaging conditions such as optical conditions. The image acquisition controller 11 controls each functional unit so that the electron beam is irradiated onto the sample 40. By configuring each functional unit, the detector is configured for forming the image, the focus position of the electron beam is adjusted, the scanning process is adjusted, and so on. The image former 12 creates an observed image at the reference position 43.

### (FIG. 5: Step S502)

The target area identifier 23 identifies, within the observed image acquired in S501, an area (referred to as target area) where inspection is performed using the feature. There exists a portion, within the observed image, that is unnecessary when calculating the feature of the inspection target. An image area excluding such unnecessary portions from the observed image is identified as the target area. A specific example of this step will be described later.

### (FIG. 5: Step S503)

The image corrector 21 corrects an image quality of the observed image. For example, an overall contrast of the observed image is intensified such that the feature of inspection target particle is more intensified. This step may be performed before S502 (FIG. 1 shows such example). A specific example of this step will be described later.

### (FIG. 5: Step S504)

The feature extractor 22 extracts an image feature of the target area identified in S502. The image feature may be a number of inspection target particles, or may be a numerical number that has a meaning equivalent to the number of inspection target particles. A specific example of feature will be described later. For example, an image feature can be calculated by extracting an inspection target particle from the observed image using image segmentation. The image feature may be calculated from a statistical amount such as histogram of brightness of image pixel, for example. Other appropriate methods may be employed.

### (FIG. 5: Steps S505-S508)

The inspection device 1 extracts an image feature of the target area for the inspection position 42 as in S501-S504.

### (FIG. 5: Step S509)

The feature comparator 25 compares the image feature at the reference position 43 with the image feature at the inspection position 42, thereby determining whether there is a significant difference between those image features. An evaluation value usable for determining whether there is a significant difference may be a P-value of t-test, for example. The P-value is calculated between the features. If the P-value is sufficiently small (e.g. P-value<0.01), it is conceivable to determine that there is a significant difference.

### (FIG. 5: Step S509: Calculation Example)

In the example shown in FIG. 2, it is determined whether there is a significant difference using a P-value between a group of 6 pieces of features acquired at the reference position 43 and a group of 6 pieces of features acquired at the inspection position 42.

### (FIG. 5: Step S510)

The reliability evaluator 26 calculates a reliability of image feature at the inspection position 42 according to the result in S509. According to the parameters calculated in S509, a calculating procedure is employed such that the reliability is higher as the significant difference is larger. However, it is not necessary to use a continuous amount as the reliability. For example, if the significant difference is at or above a threshold, the reliability is a high fixed value (e.g. 1), and if the significant difference is below the threshold, the reliability is a low fixed value (e.g. 0). In other words, any form of reliability may be employed as long as it is possible to determine whether the image feature at the inspection position 42 is appropriate for inspection.

### (FIG. 5: Step S511)

The reacquisition determiner 27 compares the reliability calculated in S510 with a threshold, thereby determining whether it is necessary to reacquire the observed image. If the reliability is at or above the threshold, the flowchart proceeds to S513. If the reliability is below the threshold, the flowchart proceeds to S512. This step is for stopping to acquire the observed image at the time when the reliability reaches the threshold, thereby providing a restriction so that the observed image is acquired only as much as necessary.

### (FIG. 5: Step S512)

The reacquisition determiner 27 checks the number of acquired observed images, thereby determining whether it is necessary to reacquire the observed image. If the number of acquired observed image is at or above a predetermined number, the flowchart proceeds to S513. Otherwise the flowchart returns to S501 and the observed image is reacquired. This step is for restricting from acquiring too many observed images even if a sufficient reliability is not acquired.

### (FIG. 5: Step S513)

The reliability displayer 33 displays the reliability calculated in S510. A display example will be described later.

### (FIG. 5: S501-S508: Additional Note 1)

This flowchart firstly acquires the observed image at the reference position 43, and then acquires the observed image at the inspection position 42. However, it is possible to firstly acquire the observed image at the inspection position 42, and then acquire the observed image at the reference position 43. In this case, S505-S508 are performed in advance, and then S501-S504 are performed. Observed images at each position may be acquired in advance, and then image features may be extracted after acquiring each observed image. It also applies to an embodiment 2 below.

### (FIG. 5: S501-S508: Additional Note 2)

In immunochromatography inspection kit, it is desirable if the state of the inspection position 42 is stable as far as possible. Therefore, the observed image at the reference position 43 may be acquired in advance, thereby keeping a time for sufficiently drying so that the state of the inspection position 42 is stable. It also applies to the embodiment 2 below. In this case, S501-S508 are performed in the sequence as described in FIG. 5.

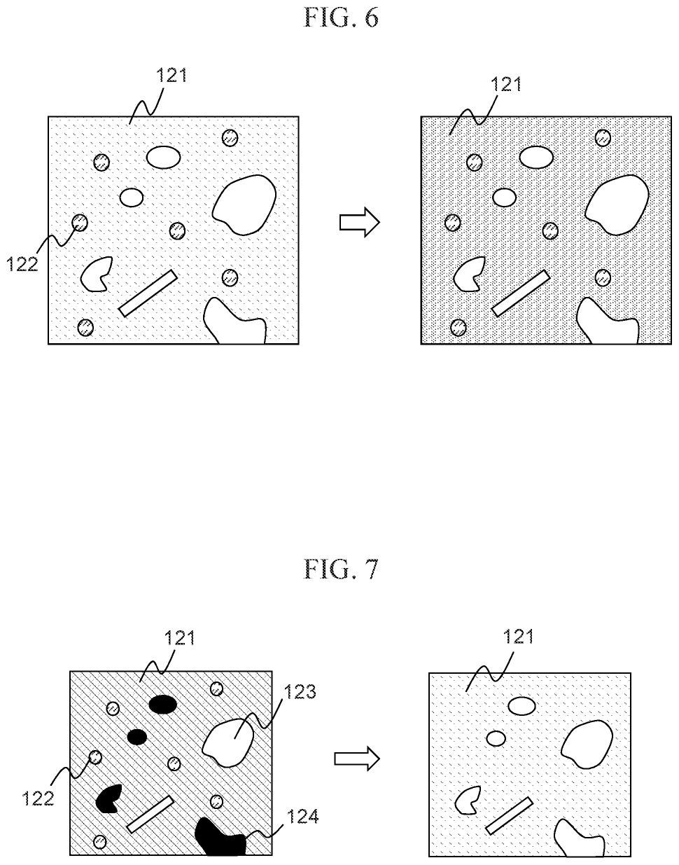

FIG. 6 illustrates a specific example of S503. If an image contrast is low between a target area 121 and an inspection target particle 122, the image corrector 21 adjusts an overall contrast of the observed image. Accordingly, as shown in the right diagram of FIG. 6, the contrast is clear between the target area 121 and the inspection target particle 122. Then it is possible to precisely acquire the feature of the inspection target particle 122. It is desirable to perform the correction onto both of the inspection position 42 and the reference position 43. However, it is conceivable to correct a contrast of one of those positions using a contrast of another one of those positions as a reference. In addition, when correcting the contrast of the observed image, a correction may be performed considering irradiating condition of the electron beam or influences on the image due to such irradiating condition.

FIG. 7 is a schematic diagram that explains a specific example of S502. Within the observed image, other than the inspection target particle 122, there may exist a portion that is unnecessary for calculating feature of the inspection target particle 122, such as an area 123 whose feature is hidden or an area 124 where no information exists. The target area identifier 23 identifies, as the target area 121, an image area where such unnecessary portions are removed from the observed image.

The area 123 is a closed area whose brightness is large and whose size is large. An example of closed area includes a large foreign object. The area 124 is, for example, a void of porous material included in the plate 41 or an image artifact. An example of image artifact includes an electrical charge by irradiation of electron beam, damage, morphological change due to vacuum evacuation, or brightness adjustment error of image.

The area 123 is formed by a foreign object whose size is large. The brightness of the area 123 may be relatively high or low compared to other areas in the observed image. This example shows a case where the brightness is high. If the area 124 is a void, its brightness is lower than that of other areas. Therefore, the target area 121 can be identified as an area, in the observed image, whose brightness is within a predetermined range (between upper limit and lower limit). The target area identifier 23 identifies the target area 121 using this method. FIG. 7 right diagram shows a result in which the target area 121 is identified by removing the areas 123 and 124.

Other method for identifying the target area 121 may be learning an unnecessary portion other than the target area 121 such as the areas 123 or 124 in advance by machine learning, and removing the unnecessary portion using the learned result. For example, it is possible to identify them by learning features of the unnecessary portion such as size, shape, or brightness. Other appropriate method may be employed to identify the target area 121.

The feature extractor 22 may extract, instead of the number of the inspection target particle 122 or along with it, features equivalent to the number of particles. For example, the feature extractor 22 may extract features such as area size or density of the inspection target particle 122. A ratio of them to area size of the target area 121 may be employed as a feature. In this case, the feature may be calculated by the equation: image feature=(feature of inspection target particle 122)/(area size of target area 121). The feature of the inspection target particle 122 may be, for example, number or area size of the inspection target particle 122. This feature is referred to as an occupied ratio (derived feature) in the embodiment 1. The occupied ratio may be calculated by the feature extractor 22, or other functional unit may be provided for calculating the occupied ratio such as the occupied ratio analyzer 24.

The feature extractor 22 extracts image features of the inspection position 42 and of the reference position 43 using a same method. For example, when using the equation above, the feature extractor 22 extracts image features by the equation above at each position, and compares the features in S509.

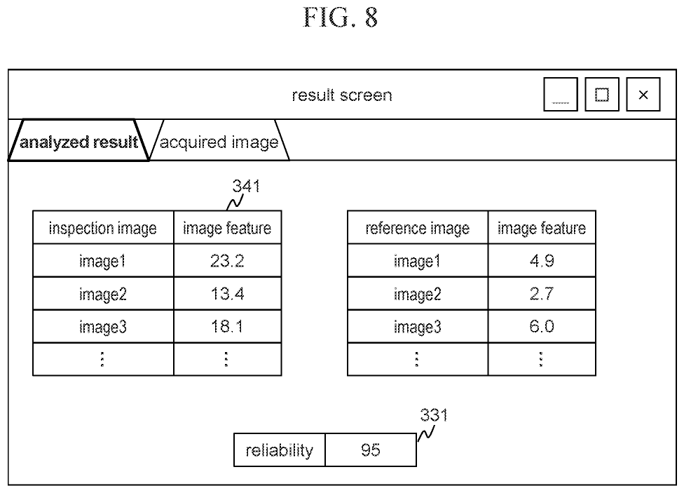

FIG. 8 is an example of user interface provided by the input/output device 30. The feature displayer 34 displays, in the display section 341, image features of each observed images acquired at the inspection position 42 and image features of each observed images acquired at the reference position 43. The reliability displayer 33 displays, in the display section 331, a reliability of image feature of observed image at the inspection position 42. This user interface may be configured by acquiring each value calculated by the evaluating device 20 and by displaying it on a display, for example.

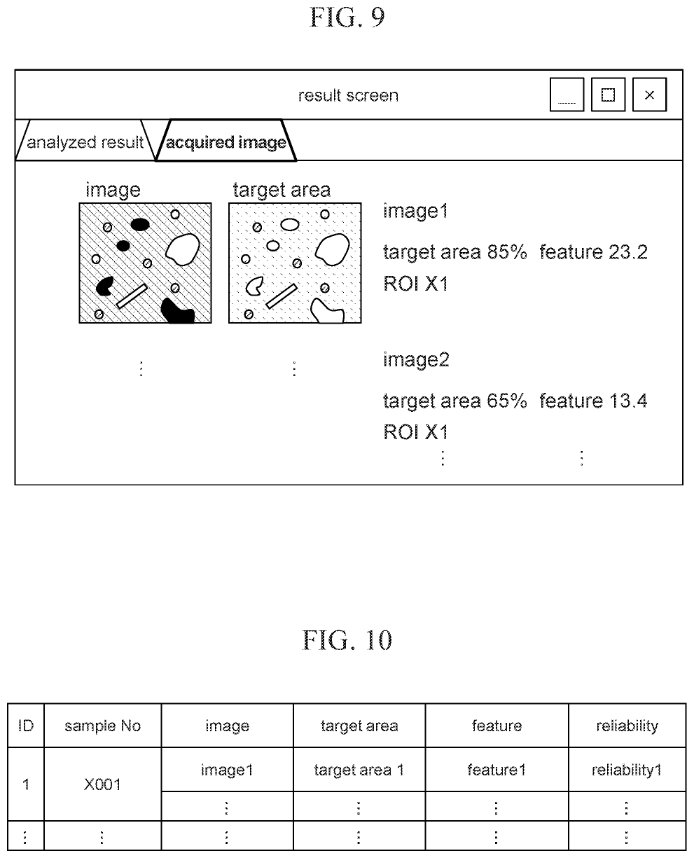

FIG. 9 is an example of user interface provided by the input/output device 30. The image displayer 31 displays each observed image acquired by the image acquiring device 10 as shown in FIG. 9. It may be possible to present, along with the observed image, information such as the target area 121 or its proportion.

FIG. 10 is an example of data stored in the storage 28. The storage 28 stores each data acquired by the evaluating device 20. For example, the data includes an observed image of the sample 40, an image of the target area 121, a feature of the observed image, or a reliability.

### Embodiment 1: Summary

The inspection device 1 according to the embodiment 1 compares an image feature of the inspection position 42 with an image feature of the reference position 43, thereby calculating a reliability of the image feature of the inspection position 42. Accordingly, it is possible to determine, in advance, whether the observed image of the inspection position 42 is appropriate for inspecting the inspection target. Therefore, if the observed image is not appropriate for inspection, it is possible to take measures such as reacquiring the observed image.

The inspection device 1 according to the embodiment 1 calculates an evaluation value (e.g. P-value of t-test) that represents whether there is a significant difference between the image feature of the inspection position 42 and the image feature of the reference position 43, thereby calculating a reliability of image feature of the inspection position 42. Accordingly, it is possible to exclude observed images that are not appropriate as the reference image. Therefore, it is possible to improve inspection accuracy.

### Embodiment 2

An embodiment 2 of this disclosure describes another behavior example of the inspection device 1. The configuration of the inspection device 1 is same as that of the embodiment 1. Hereinafter, a difference of operational procedure will be mainly described, and matters that are common with the embodiment 1 will be omitted.

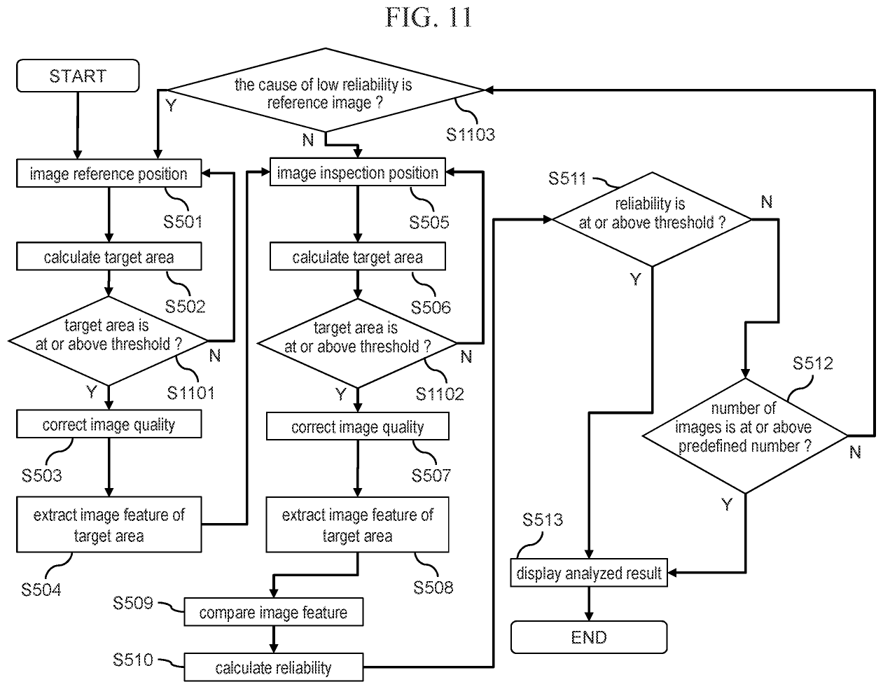

FIG. 11 is a flowchart that explains a behavior of the inspection device 1 according to the embodiment 2. S1101 is added between S502 and S503. S1102 is added between S506 and S507. S1103 is added before returning from S512 to S501. Other steps are same as those in FIG. 5.

### (FIG. 11: Steps S1101, S1102)

The target area identifier 23 determines whether the size of the target area 121 identified in S502 is at or above a threshold. If the size of the target area 121 is at or above the threshold, the flowchart proceeds to S503. If the size is below the threshold, the flowchart returns to S501 and reacquires the observed image. Depending on the method for identifying the target area 121, the target area 121 may be too small. For example, such case occurs when very large foreign object is included in the observed image. If the target area 121 is too small, it may not be possible to appropriately acquire the feature of the inspection target particle 122. Then this flowchart reacquires the observed image in such cases. S1102 similarly processes the inspection position 42.

### (FIG. 11: Step S1103)

The reacquisition determiner 27 determines whether the cause of low reliability is due to the observed image at the reference position 43. If the reference image is the cause, the flowchart returns to S501 and reacquires the observed image of the reference position 43. Otherwise the flowchart returns to S505 and reacquires the observed image of the inspection position 42. If the flowchart returns to S501, the observed image of the inspection position 42 is also reacquired. The reference image could be the cause of low reliability because the number of reference image does not reach a minimum number, because the numerical value of the reference image feature is too large, or the like.

### (FIG. 11: Step S103: Additional Note)

This flowchart firstly acquires the reference image, and then acquires the inspection image. It is also possible to firstly acquires the inspection image, and then acquires the reference image. Even in such case, if it is determined that the reference image is the cause of low reliability, the flowchart returns to the step for reacquiring the reference image.

### Embodiment 3

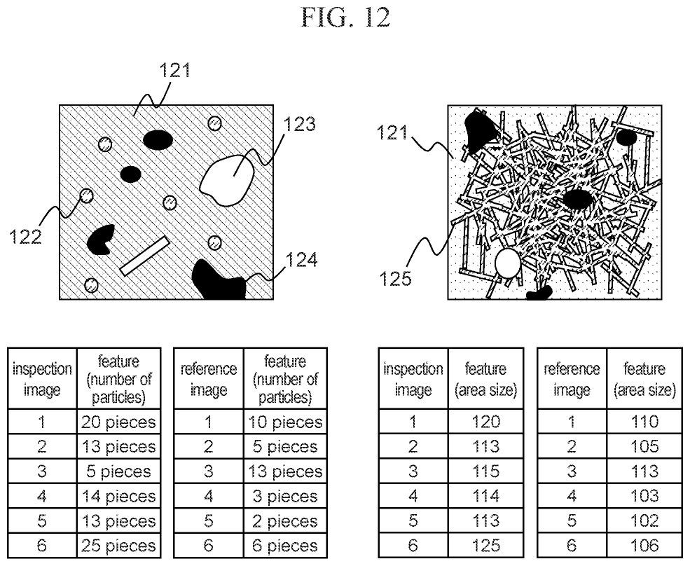

FIG. 12 is a diagram illustrating another example of the sample 40 and its image feature. An example will be described, using FIG. 12, for a relationship between a type of the sample 40 and image features.

FIG. 12 left diagram is an example of observed image using the immunochromatography inspection kit in the embodiment 1 as the sample 40. In this example, the feature at the inspection position 42 and the feature at the reference position 43 are small, respectively.

FIG. 12 right diagram is an example of observed image when using a fiber on a plate as the sample 40. In other words, the inspection target 125 included within the observed image is a fiber. In this example, it is an inspection objective to count a number of the fiber. The image feature may be the number of fiber or the area size of fiber. In this example, the fiber is dispersed to whole of the plate, and the number of the fiber is large. Therefore, the numerical value of feature is large both at the inspection position and at the reference position. Even in this case, a statistically significant difference may occur between image features at each position. Therefore, it is possible to determine whether there is a significant difference between the image feature at the inspection position and the image feature at the reference position, using the methods described in the embodiments 1-2. In other words, as long as a significant difference occurs between image features at each position, any type of the sample 40 can be inspected.

### Modification of Present Disclosure

The present disclosure is not limited to the embodiments as described above, but includes various modifications. For example, the embodiments are described in detail for readily understanding of the present disclosure which is not necessarily limited to the one equipped with all structures as described above. It is possible to replace a part of the structure of one embodiment with the structure of another embodiment. The structure of one embodiment may be provided with an additional structure of another embodiment. It is further possible to add, remove, and replace the other structure to, from and with a part of the structure of the respective embodiments.

In the embodiments above, a user may specify a specific position on the sample 40 on the user interface provided by the input/output device 30, and then an observed image is acquired at the specified position. In such case, the selector 32 receives the specified position, and instructs the image acquisition controller 11 to acquire an observed image at the specified position.

In the embodiments above, an example is described where the image acquiring device 10 and the evaluating device 20 and the input/output device 30 are a part of the inspection device 1. These devices may be separated from each other. For example, each device is configured as individual devices and is connected to each other via network, thereby it is possible to construct a configuration as in the embodiments above.

In the embodiments above, at least a part of each functional unit in the evaluating device 20 may be configured integrally. For example, the reliability evaluator 26 may include the feature comparator 25 and the occupied ratio analyzer 24. It also applies to other functional units.

In the embodiments above, it is possible to construct the inspection device 1 according to this disclosure by installing, into an existing one of the inspection device 1, software implementing the functionality of the evaluating device 20 according to this disclosure.

In the embodiments above, the inspection target by the inspection device 1 may be, for example, antigen or antibody including virus or bacillus. When using immunochromatography inspection kit for inspecting antigen or antibody as the sample 40, the component of the sample 40 includes particle, void, base material, liquid, or the like. The component of the sample 40 is not limited to such examples.

### REFERENCE SIGNS LIST

1: inspection device
10: image acquiring device
20: evaluating device
21: image corrector
22: feature extractor
23: target area identifier
24: occupied ratio analyzer
25: feature comparator
26: reliability evaluator
27: reacquisition determiner
28: storage
30: input/output device

1. An inspection device that inspects a sample using an observed image of the sample, comprising:
a feature extractor that extracts a feature of the observed image; and
a reliability evaluator that evaluates a reliability of the feature,
wherein the feature extractor extracts, within the observed image, a first feature from a target area including an inspection target,
wherein the feature extractor extracts, within the observed image, a second feature from a reference area other than the target area, and
wherein the reliability evaluator compares the first feature with the second feature, thereby calculating the reliability of the first feature.

2. The inspection device according to claim 1,
wherein the feature extractor calculates, as the first feature, a first statistical amount of a pixel value of the observed image in the target area,
wherein the feature extractor calculates, as the second feature, a second statistical amount of a pixel value of the observed image in the reference area,
wherein the reliability calculator compares the first statistical amount with the second statistical amount, thereby calculating an evaluation value that represents whether there is a significant difference between the first feature and the second feature, and
wherein the reliability evaluator calculates the reliability using the evaluation value.

3. The inspection device according to claim 1,
wherein the reliability evaluator calculates the reliability so that the reliability is higher as a significant difference between the first feature and the second feature is larger.

4. The inspection device according to claim 1, further comprising a reacquisition determiner that determines whether it is necessary to reacquire the observed image after the reliability evaluator evaluates the reliability,
wherein the reacquisition determiner determines that it is necessary to reacquire the observed image if the reliability is not at or above a reacquisition threshold,
wherein if it is determined that it is necessary to reacquire the observed image, the feature extractor re-extracts a feature of the reacquired observed image, and
wherein if it is determined that it is necessary to reacquire the observed image, the reliability evaluator reevaluates the reliability of the re-extracted feature.

5. The inspection device according to claim 4,
wherein even if the reliability is not at or above the reacquisition threshold, the reacquisition determiner determines that it is not necessary to reacquire the observed image if a number of the reacquired observed image is at or above a predetermined value.

6. The inspection device according to claim 4,
wherein if a cause of the reliability not being at or above the reacquisition threshold is due to shortage of the observed image at the reference area, the reacquisition determiner determines that it is necessary to reacquire the observed image in the reference area,
wherein if it is determined that it is necessary to reacquire the observed image in the reference area, the feature extractor re-extracts the second feature from the reacquired observed image in the reference area, and
wherein if it is determined that it is necessary to reacquire the observed image in the reference area, the reliability evaluator calculates the reliability by comparing the re-extracted second feature with the first feature.

7. The inspection device according to claim 6,
wherein if a cause of the reliability not being at or above the reacquisition threshold is due to shortage of the observed image at the reference area, the reacquisition determiner reacquires the observed image in the target area after reacquiring the observed image in the reference area.

8. The inspection device according to claim 1,
wherein the feature extractor extracts, as a derived feature of the observed image, a parameter representing a ratio of pixels of the observed image in the target area that have the feature to an area size of the target area.

9. The inspection device according to claim 8,
wherein the reliability evaluator compares the derived feature in the target area with the derived feature in the reference area, thereby calculating the reliability of the first feature.

10. The inspection device according to claim 1, further comprising a target area identifier that identifies the target area from the observed image,
wherein the target area identifier uses at least one of a brightness value of the observed image or a size of a closed area included in the observed image, thereby determining which part of the observed image is the inspection target and which part of the observed image is not the inspection target, and
wherein the target area identifier identifies, as the target area, an area excluding a portion that is determined as not being the inspection target within the observed image.

11. The inspection device according to claim 1, further comprising a corrector that corrects at least one of the observed image in the target area or the observed image in the reference area.

12. The inspection device according to claim 1, wherein the sample includes at least one of antigen or antibody.

13. The inspection device according to claim 12, wherein the feature extractor extracts, as a feature of the observed image, a number of marker particles adhered to the antigen or to the antibody.

14. The inspection device according to claim 1, wherein the inspection device is configured as a device that acquires the observed image by a charged particle beam.

15. The inspection device according to claim 1, wherein the sample includes at least one of particle, void, base material or liquid.
